(19) United States
(12) Patent Application Publication
(10) Pub. No.: US 2012/0129153 A1
(43) Pub. Date: May 24, 2012

(54) Diagnostic method for the prediction of the development and control of the effectiveness of the treatment of oncological illnesses

(76) Inventors: Artur Martynov, Kharkov (UA); Boris S. Farber, Brooklyn, NY (US); Sonya Sophya Farber, New York, NY (US)

(21) Appl. No.: 12/931,462

(22) Filed: Feb. 1, 2011

Related U.S. Application Data
(63) Continuation of application No. PCT/RU2010/000696, filed on Nov. 22, 2010.

(51) Int. Cl. C12Q 1/70

(52) U.S. Cl. 435/5

(57) ABSTRACT

A diagnostic method, in which patient tissue samples are taken, microassay are prepared, specific anti-viral immunoglobulins are processed, the number of cells infected by two or more viruses before the beginning of treatment are determined, and the dynamic of the change in the number of infected cells and their interrelationships are established: if the number of blood cells infected by any two or more viruses exceeds 50±10% in patients without signs of oncological pathology, a diagnostic conclusion is drawn of a high danger of oncological illness in connection with immune system ineffectiveness; if the number of cells infected by any two or more viruses exceeds 50±10% in patients with diagnosed oncological illnesses, a diagnostic conclusion of the cancer tumor's low sensitivity to chemotherapy and the perspective of quick tumor metastasis is drawn.

### TECHNICAL FIELD

This invention is related to medicine—specifically, to therapy and oncology—and is designated for the prediction of the development and effectiveness control of the treatment of oncologic diseases.

### Previous Level of Technology

Which organisms benefit from infected cells going out of immune control? It is the intracellularly persistent agents that only transition into the active reproductive phase a few times a year and lie dormant inside the infective cells the rest of the time that are good for blocking immune response. The most widespread viruses from the herpesvirus, flu virus, papillomavirus, and adenovirus, persistent intracellular microorganisms like mycoplasms, chlamydia, legionelles, and many other intracellular infective agents are types of persistent agents. The basic mechanism that prevents the liquidation of the infected cells by the immune system is the blocking of apoptosis through a variety of mechanisms.

In many cases, the viral persistence leads to the cancerous transformation of target cells. Like chemical carcinogenesis, this viral carcinogenesis is a multi-stage process. Cell transformation begins after infection of oncogenic viruses. In in vitro experiments, the next stage is characterized by the formation of a colony of cells with changed structure and multi-layered growth. These cellular properties are irreversible, and in many cases are not dependent on the presence of carcinogenic viruses inside them. As a rule, these cells may form in vivo tumors after subinoculation into syngen or athymus animals.

Carcinogenic viruses that are capable of playing the role of tumor-generating agents are represented as DNA and RNA viruses. For transformation of cells by DNA viruses an integration of the viral genome into the cell genome and the transfer of the changed genetic information to the cell's descendants must take place. Carcinogenic RNA viruses can also integrate their genomes into cell chromosomes, but initially, proviral DNA is synthesized with the participation of revertase in the infected cell in the RNA matrix. The synthesis of cell DNA is a required condition for the integration of viral DNA with cellular genes. The basic genes of carcinogenic viruses—viral carcinogens—take part in the initiation and support of the malignant transformation of cell targets. Upon stable transformation of cells, the carcinogen attaches itself to the genome and is in its activated state, while if there is an abortive transformation, the carcinogens function, but are not necessarily retained in the cell. The transformative effect of carcinogens is conditioned on the pleiotropic action of the proteins that are coded by these genes. This action may stimulate anomalously intensive cell division and (or) the blocking of cell apoptosis.

The discovery of endogenic viruses that are components of the genome of normal cells permitted a new interpretation of L. A. Zilber's viral genetic theory [¹]. However, questions on the meaning of the presence of endogenic viruses in the repressed (persistent) form and the possibility of their vertical transfer to descendants have not yet been answered. In connection with this, it is important to note the possibility of recombination between exo- and endogenic carcinogenic viruses, which allows defective sarcomatous viruses to complete their own reproductive cycle.

In the evolutionary process, two protective mechanisms formed in mammals that control the elimination from the organism of cells infected by viruses. The first of these is based on the immune reaction of the organism, which is directed against foreign viral proteins. This reaction manifests in the cytotoxic effect of special immune cells, which leads to the elimination of cells that have been transformed or infected by the virus. Cytotoxic T-lymphocytes fulfill their effector functions with the assistance of Fas-ligand/Fas receptor systems and/or granzyme-perforin systems [²]. Another protective mechanism is connected with the activation of the cell cycle by the viral proteins in the infected cells [³]. Unlike that induced by cytokines, this activation ends in cell death through apoptosis [⁴].

The participation of the Epstein-Barr virus (EBV) has been established in the development of infectious mononucleosis and association with lymphoproliferative illnesses, nasopharyngeal and stomach cancers, and certain lymphomas, including Burkitt's lymphoma. In the last case, the virus infects circulatory B-lymphocytes exclusively [⁵], and the increased survivability of these cells is an important factor in the persistence of the virus. The significance of the EBV in the formation of a malignant phenotype in lymphoma cells was demonstrated not long ago by J. Komano et al [⁶]. EBV—the positive cell line for Burkitt's lymphoma was selected by EBV—has negative clones that later underlay the infection by this virus. As it turns out, these clones (unlike the non-infectious EBV) are capable of growing in a semisolid agar medium and causing tumors in animals. The EBV-positive clones of the Burkitt's lymphoma cells demonstrated a significantly higher resistance to apoptosis than did the EBV-negative clones. Based on these data, the authors came to the conclusion that in order for the development of a malignant phenotype and resistance to apoptosis, the constant presence of EBV is required in Burkitt's lymphoma cells.

Testing of the action of the antiviral drug Cidofovir on the duplication of EBV-associated nasopharyngeal carcinomas in athymous mice established that this drug causes a quick induction of apoptosis in EBV-transformed epithelial cells [⁷]. The DNA of EBV appears in the blood serum of nasopharyngeal carcinoma patients [⁸], which also bears witness to its connection to the development of this illness.

The study of the molecular mechanisms of EBV-induced carcinogenesis presented the opportunity to discover a group of apoptosis-inhibiting proteins that take part in the formation and further development of the tumor process. The incipient EBV antigen complex BHRF1 stimulates the B-lymphocytes that are infected by EBV to undergo the cellular cycle and survive [⁹], which can increase the tendency of infected cells toward malignant transformation. Another protein, LMP1, which is coded by the EBV, demonstrates properties of a receptor that is capable of activating anti-apoptosis genes Bcl-2 and A20. It has been established that the anti-complementary oligonucleotides against the LMP1 gene slow proliferation, stimulate apoptosis, and increase the sensitivity of the B-lymphocytes immortalized by the EBV to the action of chemotherapy drugs [¹⁰].

Similarly to the Epstein-Barr virus, the type 1 and 2 herpes simplex viruses (HSV-1 and -2) and Herpesvirus saimiri demonstrate the properties of carcinogenic viruses. For example, it was discovered that transplantation of cells transformed by HSV-2 into athymic mice causes the formation of tumors in the animals [¹¹]. This virus demonstrates an affinity to cells in genital organs, while HSV-1 shows an affinity for mucus membranes of the lips and nasopharyngeal area, as well as to human skin integuments. It has been established that there is DNA fragmentation in cells infected by a mutated form of HSV-1 that lacks the α4 and Us3 genes (which code the main regulatory protein and the viral protein kinases respectively), while the “wild” (mutation-free) type of the virus does not have this effect [¹²]. Moreover, the “wild” type of the virus blocks human neuroblastoma cell apoptosis induced by the α-tumor necrosis factor using antibodies against Fas receptors, ceramides, or hyperthermia. A tissue specificity exists for the anti-apoptotic action of HSV-1 proteins. For example, human epidermal uterine carcinoma cells are resistant to this influence. The fact that the blockade of apoptosis of HSV-1-infected cells is not connected to its active reproduction is very important [¹³]. Unlike HSV-1, HSV-2 is capable of slowing the activity and level of Fas-ligand expression in a cell membrane [¹⁴]. The infection of T-cells leads to the Fas-ligand remaining hidden in the cell and not being expressed on cell plasmalemma. As a result, these cells lose that cytotoxic activity which is facilitated through Fasdependent apoptosis.

The other carcinogenic virus, Herpes saimiri, codes protein ORF16, which is a functional analogue o the Bcl-2 protein [¹⁵]. As it turns out, a viral protein similar to Bcl-2 may create heterodimers with the pro-apoptotic Bak and Bax proteins, which results in the blocking of apoptosis induced by heterological viruses. For certain α-herpesviruses (and the verrucas planae virus), the production of special vFLIP anti-apoptotic proteins capable of cooperating with the FADD cell adaptor protein is inherent [¹⁶]. The survival of cells infected with these viruses facilitates the constant influence of interfering carcinogenic viruses that increase their transforming potential to a significant extent.

The tumor forming activity of the human cytomegalovirus (HCMV) was recently demonstrated in vitro in experiments on primary cultures of the kidneys of embryonic rats [¹⁷]. The cancerous transformation of the cells evoked early HCMV genes IE1 and 1E2, which, in combination with the gene from the E1A adenovirus, activated mutation in cell genes. It was proven that the products of the viral IE1 and IE2 genes can block apoptosis independently of one another [¹⁸]. IE proteins fulfill the function of transcription factors; the anti-apoptotic function of the IE2 protein is connected with the activation of the expression of cycline E (which is responsible for cells' transition to G1 in the S-phase of the cellular cycle) and the slowing of the post-transcription activity of the p53 protein [^{19, 20}].

L. Burns et al [²¹] have established that the IE1 and IE2 viral proteins synergetically activate the expression of the ICAM-1 intercellular adhesion molecules in endothelial cells. The infection of neuroblastomal cells by the cytomegalovirus was accompanied by changes in their cytoskeletons and the level of expression of the integrated receptors, which increased the mobility of the cells and their dissemination [²²]. It is interesting that in long-term culturing of neuroblastomal cells infected by HCMV, the developed a resistance to the action of cysplatin and etoposide, although the viral DNA was not distinguished in them [19]. When the reproduction of the virus was blocked through treating the cells with Ganciclovir, their sensitivity to the action of anti-tumor drugs was fully restored. These data indicate that the infection of cells with cytomegalovirus before or in the process of tumor growth may increase their survival and the development of resistance to the action of anti-tumor drugs. Thus the inclusion of antiviral drugs in the scheme for the treatment of malignant tumors and cardiovascular disease is justified.

The nature of nosotropic changes in the body of herpes patients is conditioned to a significant extent on the ability to integrate the virus's genome with the cell carrier genome, in part in the paravertebral ganglions, and on the affinity of HSV and other herpesviruses to blood corpuscles (erythrocytes, thrombocytes, granulocytes, and macrophages) and immunocytes [23]. This facilitates the life-long persistence of HSV in the human body and provides the conditions for changes to the cellular and humoral immune systems. Moreover, HSV is an infectious (acquired) disease of the immune system [24], in which the long-term persistence of the virus in many cases is accompanied by abortive HSV infections in nearly all types of immune system cells, which is often accompanied by their functional inadequacy and facilitates the creation of immune deficit [25,26]. It has been proven by many researchers that a major role in the formation of immunity to herpes belongs to cell mechanisms, whose condition is largely determined as a result of both initial infection and frequency and duration of relapses [²⁷]. The duration of an immune deficit under circumstances of viral infections depends on both the properties of the virus itself and on the type of corresponding reactions exhibited by the patient. The fairly large level of specific antibodies in the blood of patients with recurring herpes stabilizes the persistence of the virus, but does not anticipate relapses [28]. A good deal of research [29,30] indicates that in a severe herpesvirus infection (SHVI), the total number of CD3+ (general T-lymphocytes) and CD4+ (T-helper) cells is decreased, as is the immunoregulatory balance (CD4+/CD8+). There is decreased activity in natural killers and antibody-dependent cell toxicity and a slowed ability of the leucocytes to synthesize endogenous interferon. These changes are characteristic of patients with severe HSV infections in the moderate or severe stages of the disease. It has also been established that B-cells respond more actively to pyrogenal than T-lymphocytes react to tuberculin, which confirms the functional inadequacy of the T-cell component in the immune system of the patients being studied. In the remission phase, a raised level of circulating immune complexes is observed, which bears witness to the inadequacy of monocyte-macrophage cells, since the herpes virus has an affinity to these cells and can destroy them [31].

We may therefore conclude that in recurring severe herpesvirus infections, the T- and B-cell immune systems from which the deciding role in antiviral protection (and in the case of the T-cell system, antitumor protection) are inhibited and brought out of balance. A functional deficit of many non-specific resistance factors in the body is also seen [32]. The disruptions to immune homeostasis discovered are registered in the herpesvirus infection relapse and remission phase, largely directing the development of the long-term persistence of the herpesvirus in the body with the establishment of a relapsing course to the illness [33].

If a mutated or tumor cell appears in a body with this immune condition, that cell will not be recognized and destroyed. Correspondingly, a chronic, hidden immune deficit may facilitate the immune system's insensitivity to malignant tumors. HSV's persistence in lymphocytes will lead to the expression and introduction into the histocompatibility complex of denatured viral proteins and immature Di-particles (defective interference particles), to which a large quantity of the immunoglobulins making up the immune complexes joins. These complexes form on both the membranes of cancer cells (herpesvirus-infected cells) and on the membranes of infected immunocytes, blocking the reaction of the recognition of the changed histocompatibility complex of cancer cells.

Correspondingly, the immune system does not “destroy” cancer cells and metastatic cells, and the immune modulators activate an increase in the concentration of anti-herpesvirus immunoglobulins and immune complexes with them, which worsens the imbalance of the immune system and activates cancer metastasis.

A method of detection and differentiation of stages of illnesses with a high level of specificity and sensitivity is known. The method allows us to determine the presence of changed cells in samples that contain them; it is also capable of determining the histological type of the presenting illness for certain diseases. The method dictates changes in the level and structure of expression of molecular markers in samples containing cells. A procedure for method validation and group determination has also been presented. The method is applicable for the detection of cancer cells, microbial cells, differentiation between herpesvirus infections, chlamidiosis, trichomonadiosis, and gonorrhea [³⁴]. The method's shortcoming is its lack of algorithms that allows a precise prediction of the effectiveness of treatment for cancer patients with diagnoses that have already been made and the prediction of the possibility and intensity of cancer metastasis for cancer patients and of the possibility of the development of oncological pathology according to the dynamic of the change in the quantity of cells infected with two or more viruses. The method from the prototype is designated exclusively for making initial diagnoses and does not provide algorithms for treatment control, tumor sensitivity to chemotherapy, or the prediction of disease treatment effectiveness in cancer patients with diagnoses that have already been made and confirmed earlier histologically. The method does not suggest the use of antiviral immunoglobulins for the discovery of cancer cells and immune system cells infected by viruses in the dynamic of the development of oncological illness, which does not allow the chance to predict the development of cancer in healthy people or evaluate the severity of the development of the illness in cancer patients according to the level of viral pressure on the immune system.

### DISCLOSURE OF THE INVENTION

The invention's task was to develop a diagnostic method for the prediction of the development and control of the effectiveness of the treatment of oncological illnesses that allows interactive control of the effectiveness of the treatment of cancer patients by standard methods in combination with anti-viral substances, to predict the intensity of the metastasis and sensitivity of cancer cells to chemotherapy, and to predict the danger of the appearance of cancer in practically healthy people by the level of infected immunocytes.

The task set is addressed through a diagnostic method for the prediction of the development and control of the effectiveness of the treatment of oncological illnesses, in which patient tissue samples are taken (immunocytes from venous and capillary blood, erythrocytes, cells from urinary and salivary sediment, and smears and prints of biopsied tumor material), microdrugs are prepared, specific antiviral immunoglobulins are processed (for types 1 and 2 herpes viruses, the cytomegalovirus, the herpes Zoster virus, the Epstein-Barr virus, the herpes 6 virus, or the combinations thereof), the number of cells infected by two or more viruses before the beginning of treatment, during treatment, and after treatment are determined with the application of antiviral therapy, and the dynamic of the change in the number of infected cells and their interrelationships are established: when the number of infected cells decreases by more than 20±10%, the treatment is considered successful, whereas an absence of changes or an increase in the number of infected cells is considered an indication of unsuccessful treatment; if the number of blood cells infected by any two or more viruses exceeds 50±10% in patients without signs of oncological pathology, a diagnostic conclusion is drawn of a high danger of oncological illness in connection with immune system ineffectiveness; if the number of cells infected by any two or more viruses exceeds 50±10% in patients with diagnosed oncological illnesses, a diagnostic conclusion of the cancer tumor's low sensitivity to chemotherapy and the perspective of quick tumor metastasis is drawn in connection with immune system ineffectiveness; if the method is used after conducting treatment with a combination of traditional methods and the use of antiviral drugs, changes in the percentage of infected cells are determined after treatment, and a decrease in the number of infected cells is seen in repeat diagnosis by 20±10% or more for two or more viruses, a conclusion is drawn on the effectiveness of the therapy, while if a decrease in the number of infected cells in repeat diagnosis by 20±10% for even one of the viruses diagnosed by this method, a conclusion is drawn on the ineffectiveness of the therapy applied in that period and the need to change the antiviral therapy scheme.

### SHORT DESCRIPTION OF DRAWINGS

FIG. 1—Lymphocytes Infected by the Type 1 Herpes Virus

FIG. 2—Division of Patients into Groups by Herpesvirus Persistence

FIG. 3—Division of Groups of Patients with Herpesvirus Persistence in Immunocytes by Fluorescence Index

FIG. 4—Percentage of Lymphocytotoxic Immunoglobulins in the Blood Serum of Each Patient Group

FIG. 5—The Level of Circulating Immune Complexes in the Blood Serum of Cancer Patients and a Control Group

FIG. 6—Results of the Study of the Blast Transformation of Peripheral Blood Lymphocytes of Patients under the Influence of Phytohemagglutinin

### BEST INVENTION IMPLEMENTATION OPTION

### EXAMPLE 1

As a result of the study, a correlational dependence was established between changes in the immune system of cancer patients (both with and without herpesvirus persistence) and patients with severe herpesvirus infections (both with and without herpesvirus persistence in their immunocytes). The following was planned for the study:

confirmation of the presence of HSV in various organs and tissues of the cancer patients

an analysis of the immunograms of patients from four groups: patients with persistent herpesvirus in their immunocytes (cancer patients and control group) and patients without persistent herpesvirus (also cancer patients and control group)

the establishment of characteristic signs of herpesvirus persistence through immunological test data

Cancer patients who were being treated in the cancer ward of a hospital were studied. The materials for the study were: biopsy material from the tumors and surrounding tissues, blood serum, and lymphocytes. Herpes patients with severe herpesvirus infections but without signs of oncological illness were used as a control group.

The patient group was made up of 91 patients, of which 55 were female and 36 were male. Average patient age was (52±15) years. In the control group, 42 herepes patients were studied, of which ten were male and 32 were female. Average patient age in the control group was (50±20) years. In total, 343 analyses were performed, of which 110 were in the control group.

The cancer patients studied were divided into the following groups: 28 had uterine cancer (UC); 13 had ovarian cancer (OC); 12 had mammary gland cancer (MGC); 1 had stomach cancer (SC); 11 had large intestinal cancer (LIC); 10 had lung cancer (LC). All diagnoses had been confirmed histologically. The majority of the patients had the beginnning stage of the deseases and had not previously undergone specific antitumor or antiviral treatment.

Establishment of the fact of herpesvirus persistence in immunocytes (HPI). An immunofluorescence reaction was used to determine the presence of HSV-1 in the lymphocytes. The reaction was conducted in accordance with the instructions for the test system (the test system for the reaction of direct immunofluorescence belonging to a lab diagnostics firm [Moscow, Russia] with monoclonal antibodies to the HSV-1 early protein).

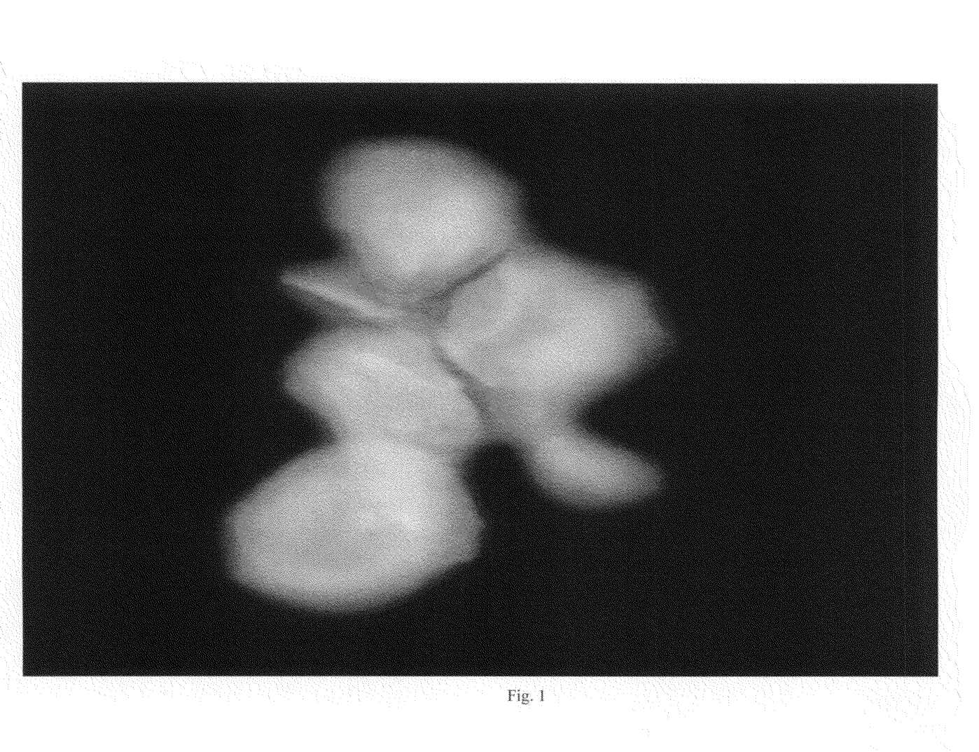

The result was considered positive in the presence of no fewer than five morphologically unchanged cells with an intense, bright green, specific glow of typical localization (FIG. 1).

The quantitatively positive results obtained during the reaction were evaluated according to the formula:

FI=(A-B)/A,

where
A is the percentage of non-fluorescing cells in the control substance;
B is the percentage of non-fluorescing cells in the experimental substance;
The quantitative reaction is considered positive if the fluorescence index comes to >0.2. The separation of lymphocytes from the peripheral blood was done on ficoll-verografin [35]. The lymphocytes and prints of their biopsy material were fixed with acetone and then processed with a FITC fluorescent marker special antiviral immunoglobulins in the concentrations indicated in the instructions. The smears were incubated over thirty minutes at a temperature of 37° C.; the remainders of the antibodies were then rinsed off and the smears were dried and studied under a luminescent microscope.

The study of immunological indicators was conducted in accordance with the standard methodologies set forth in [35,36]:

calculation of the total number of lymphocytes (absolute and relative contents in the blood)

determination of the number of T- and B-lymphocytes through a rosette reaction

evaluation of the phagocytic activity of neutrocytes

determination of the major classes of blood serum immunoglobulins (IgA, IgM, IgG) by the immunoferment method

determination of the complement's titer

determination of the circulatory immune complexes

conduct of a lymphocyte transformation (BTL) reaction with phytohematoagglutinin

It is well-known that in the majority of cases with oncological illnesses, the parameters of standard immunograms (quantitative indicators) in cancer patients do not differ from those in the control group. This fact is conditioned on the use of immune system parameters in the research that characterize quantitative changes: the number of immunocytes, the number of immunoglobulins, and others. It is well-known that an insignificant increase in circulatory immune complexes and a decrease in the phagocytic index are often observed in cancer patients. However, these data contradict each other in various references.

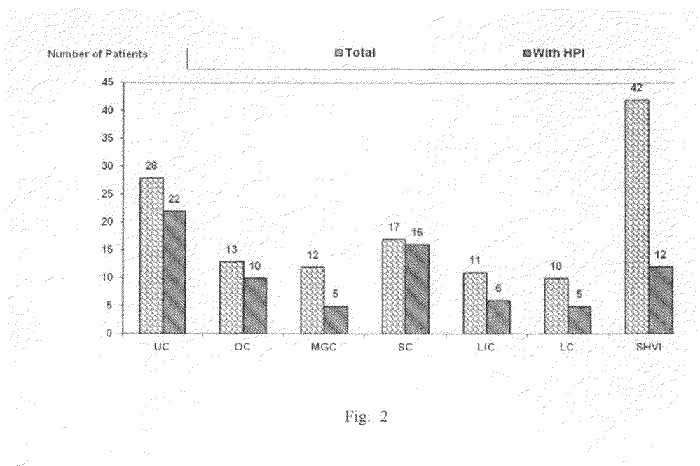

The results of the determination of the herpesvirus persistence in immunocytes by type of tumor are indicated in FIG. 2.

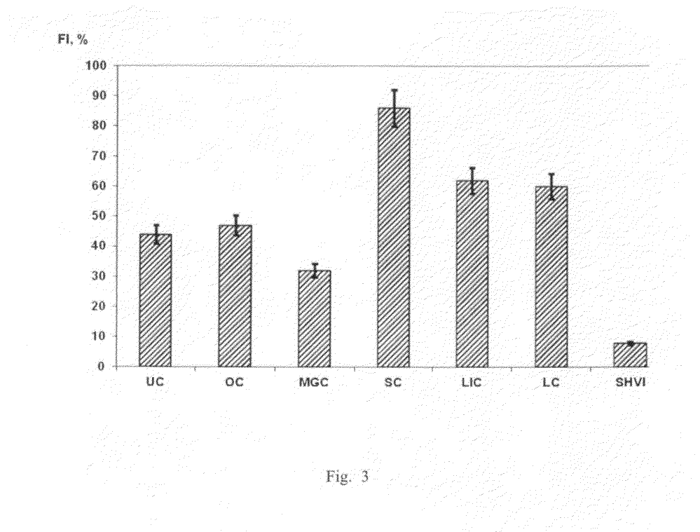

As may be seen in FIG. 2, in all groups of cancer patients with various adenocarcinomas, a significantly larger number of patients were observed with herpesvirus persistence in immunocytes than there were in patients without oncological illnesses. In the control group of patients with severe herpesvirus infections, only 12 of the 42 studied had HSV-1 in their immunocytes. Thus, on the average, the percentage of cancer patients with herpesvirus persistence was 67.4% of the total number of cancer patients in the experimental group and only 28.6% in the control group (patients with severe herpesvirus infections). The results of the study of the determination of groups with herpesvirus persistence in immunocytes according to the fluorescence index (the percentage of immunocytes infected with viruses out of their total number) are indicated in FIG. 3.

As may be seen in FIG. 3, the highest fluorescence index is seen in stomach cancer patients (86±8%). Also, more than half of the lymphocytes were infected in lung and large intestinal cancer patients (60±10 and 62±6% respectively). The smallest fluorescence index was observed in the group of patients with mammary gland cancer (32±12%). In the group of uterine and ovarian cancer patients, the average value for the fluorescence index did not reach 50% either, at (44±5)% and (47±7)% respectively. However, in all the experimental groups, the fluorescence index value exceeded the analogous indicator in the control group of patients with severe HSV, which was (8±5)%.

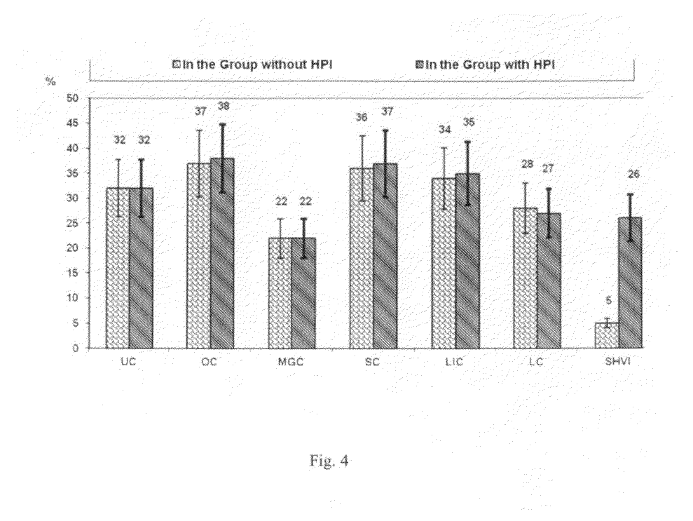

Thus the average fluorescence index indicator in the group of cancer patients was (55.2±8)%, while the control group indicator was only (8±5)%. We may therefore confirm with a high level of certainty that the herpes virus is involved in the mechanism that allows a cancerous tumor to go out of immune system control. Further, the immunological changes characteristic of the two types of patients—those with herpesvirus persistence—were studied. (Only those parameters of immunity are indicated that changed by more than ten percent.) In FIG. 4, the percentage of lymphocytotoxic immunoglobulins in the blood serum of patients is shown for each group of patients.

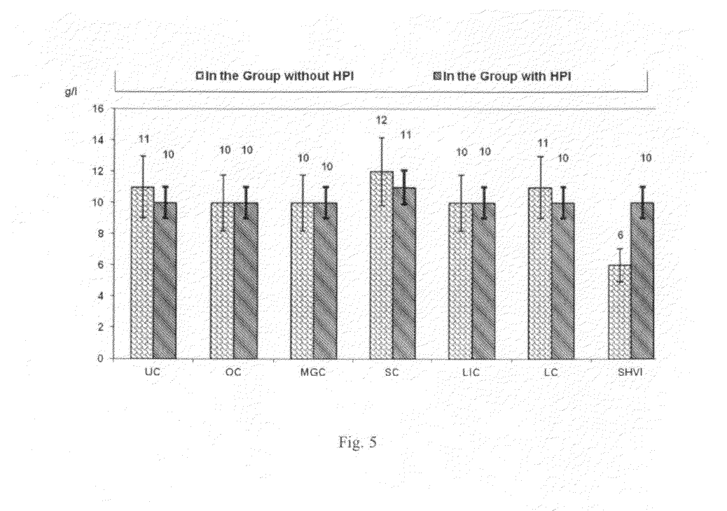

As may be seen in FIG. 4, a significant difference was not observed between the groups of cancer patients with and without herpesvirus persistence in immunocytes. A significant difference was observed only in the control group: the patients with severe herpesvirus infection without herpesvirus persistence in immunocytes had a quantity of lymphocytotoxic immunoglobulins that was one fifth of that of the group with herpesvirus persistence in immunocytes. This shows that the herpes viruses that persist in immunocytes due to the expression of viral antigens on the histocompatibility complex provokes an attack on the lymphocytes by the lymphocytotoxic immunoglobulins. The fact that a significant statistical difference was not observed between the groups of cancer patients with and without herpesvirus persistence in immunocytes is very important. Both groups were characterized by high lymphocytotoxic immunoglobulin values, which is characteristic of infected lymphocytes. In our opinion, this fact may bear witness to the persistence in the immunocytes of cancer patients (that group of patients in which herpesvirus persistence in immunocytes was not found) of another persistent virus or viruses. These could be type 7-12 herpes viruses, adenoviruses, papilloma viruses, other persistent viruses or their combinations, intracellular infections (chlamydia, mycoplasms, etc.) separately or in combination with viruses. Another immune system indicator that changed is the circulatory immune complexes. The results of the study of the dependence between the level of circulatory immune complexes in patients' blood and herpesvirus persistence in immunocytes are presented in FIG. 5.

As may be seen in FIG. 5, the level of circulatory immune complexes did not have substantial changes in the groups with or without herpesvirus persistence in immunocytes, but they were remarkable for high values characteristic of the control group with herpesvirus persistence in immunocytes. In the group of patients with severe herpesvirus infection without herpesvirus persistence in immunocytes, the level of circulatory immune complexes did not exceed (6±2) g/l. This indicates that in both groups of cancer patients—with and without herpesvirus persistence in immunocytes—a picture is seen that is characteristic to the persistence in immunocytes of some kind(s) of virus: the level of circulatory immune complexes in a group of cancer patients varied from (10±2) g/ml to (11±2) g/ml.

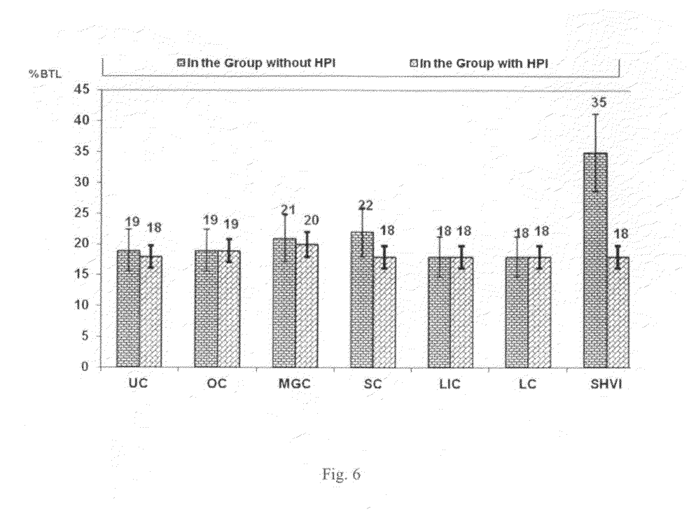

The last parameter of the immunogram that changed in all the cancer patients was lymphocyte transformation. The results of the study of changes in lymphocyte transformation in the cancer patients and the patients in the control group are presented in FIG. 6.

As may be seen in FIG. 6, between the two groups of cancer patients, there was no difference in those with and without herpesvirus persistence in immunocytes. The level of lymphocyte transformation in both groups is characteristic for viral persistence. In the control group of patients with herpesvirus persistence in immunocytes, the level of lymphocyte transformation came to (18±5)%, whereas in the control group without herpesvirus persistence in immunocytes, the level of lymphocyte transformation nearly corresponds to the norm at (35±5)%. This fact bears witness to the high likelihood that a virus persists in the lymphocytes of all the cancer patients: in some cases, this is the herpes virus; in others, they are unknown intracellular agents (other viruses, chlamydia, mycoplasms, rahnellas, or others).

As far as the other patient immunogram parameters studied (not discussed) goes, they were not statistically different from the immunograms of the control group with severe herpesvirus infection without herpesvirus persistence in immunocytes.

Thus in all groups of cancer patients with various adenocarcinomas, a significantly larger amount of patients were observed with herpesvirus persistence in immunocytes. In the control group of patients with severe herpesvirus infections, only 12 of the 42 studied had HSV-1 in their immunocytes. On the average, the percentage of cancer patients with herpesvirus persistence was 67.4% of the total number of cancer patients in the experimental group and only 28.6% in the control group (patients with severe herpesvirus infections). The largest fluorescence index was observed in stomach cancer patients (86±8%). Also, more than half of the lymphocytes were infected in lung and large intestinal cancer patients (60±10 and 62±6% respectively). The smallest fluorescence index was observed in the group of patients with mammary gland cancer (32±12%). In the group of uterine and ovarian cancer patients, the average value for the fluorescence index did not reach 50% either, at (44±5)% and (47±7)% respectively. The average fluorescence index indicator in the group of cancer patients was (55.2±8)%, while the control group indicator was only (8±5)%. A significant difference in the level of lymphotoxic immunoglobulins was observed only in the control group: the patients with severe herpesvirus infection without herpesvirus persistence in immunocytes had a quantity that was one fifth of that of the group with herpesvirus persistence in immunocytes. In the group of patients with severe herpesvirus infection without herpesvirus persistence in immunocytes, the level of circulatory immune complexes did not exceed (6±2) g/l. This indicates that in both groups of cancer patients—with and without herpesvirus persistence in immunocytes—a picture is seen that is characteristic to the persistence in immunocytes of some kind(s) of virus: the level of circulatory immune complexes in the group of cancer patients varied from (10±2) g/1 to (12+2) g/l. The level of lymphocyte transformation in both groups of cancer patients is characteristic of viral persistence. In the control group of patients with herpesvirus persistence in immunocytes, the level of lymphocyte transformation came to (18±5)%, whereas in the control group without herpesvirus persistence in immunocytes, the level of lymphocyte transformation nearly corresponds to the norm at (35±5)%. We may therefore confirm with a high level of certainty that the herpes virus is involved in the disruption of the immune antitumor response.

### INDUSTRIAL APPLICABILITY

This invention is related to medicine—specifically to oncology—and may be used in cancer clinics to improve the diagnosis and control of the effectiveness of the treatment of cancer patients with the goal of increasing the effectiveness of their treatment. All the proposed components of the diagnostic test system are produced by the pharmaceutical industry and are accessible for use.

### REFERENCE

1 Zilber L A, Irlin I S, Kisilev F L. The Evolution of the Viral-Genetic Theory of Tumor Occurrence. Moscow: Science.-1975.-344 pp.

2 Podack E. R. Execution and suicide: cytotoxic lymphocytes enforce Draconian laws through separate molecular pathways.//Curr. Opin. Immunol.-1995.-Vol. 7.-P.11-16.

3 Prokhoryatova O V, Bila N V, Volkova V V, Martinov A V. A study of the interference of infectious laryngotracheitis viruses and Newcastle Disease in chicken embryos.//Veterinary Medicine.- 2000.-vol.1, No 78.- pp. 251-255.

4 Eick D., Hermeking H. Viruses as pacemakers in the evolution of defense mechanisms against cancer. //Trend. Genet.-1996.-Vol. 12.-P. 4-6.

5 Yao Q. Y., Ogan P., Rowe M, et al. Epstein-Barr virus-infected B cells persist in the circulation of acyclovir-treated virus carriers.//Int. J. Cancer.-1989.-Vol. 43.-P. 67-71.

6 Komano J., Sugiura M., Takada K. Epstein-Barr virus contributes to the malignant phenotype and to apoptosis resistance in Burkitt's lymphoma cell line Akata.//J. Virol.-1998.-Vol. 72.-P. 9150-9156.

7 Neyts J., Sadler R., De Clercq E, et al. The antiviral agent Cidofovir [(S)-1-(3-hydroxy-2-phpsphonyl-methoxypropyl)cytosine] has pronounced activity against nasopharyngeal carcinoma grown in nude mice.//Cancer res.- 1998.-Vol. 58.-P. 384-388.

8 Matirangura A., Pornthanakasem W, Theamboonlers A, et.al. Epstein-Barr viral DNA in serum of patients with nasopharyngeal carcinoma.//Clin. Cancer Res.- 1998.-Vol. 4.-P. 665-669.

9 Dawson C. W., Dawson J., Jones R., et al. Functional difference between BHRF1, the Epstein-Barr virus-encoded Bcl-2 homologue, and Bcl-2 in human epithelial cells.//J. Virol.-1998.-Vol. 72.-P 9016-9024.

10 Kenney J. L., Guinness M. E., Curiel T., Lacy J. Antisense to the Epstein-Barr virus (EBV)-encoded latent membrane protein 1 (LMP-1) suppresses LMP-1 and bcl-2 expression and promotes apoptosis in EBV-immortalized B cells.//Blood.-1998.-Vol. 92.-P. 1721-1727.

11 Aurelian L. Herpes simplex virus type 2: unique biological properties include neoplastic potential mediated by the PK domain of the large subunit of ribonucleotide reductase.//Front Biosci.- 1998.-Vol. 3.-P. 9237-9249.

12 Galvan V., Roizman B. Herpes simplex virus 1 induces and blocks apoptosis at multiple steps during infection and protects cells from exogenous inducers in a cellOtype-dependent manner.//Proc. Natl. Acad. Sci. USA.-1998.-Vol. 95.-P. 3931-3936.

13 Koyama A. H., Miwa Y. Suppression of apoptotic DNA fragmentation in herpes simplex virus type 1-infected cells.//J. Virol.-1997.-Vol. 71.-P. 2567-2571.

14 Sieg S., Yildirim Z., Smith D, et. al. Herpes simplex virus type 2 inhibition of Fas ligand expression.//J. Virol.-1996.-Vol.70.-P.8747-8751.

15 Nava V. E., Cheng E. H., Veliuona M, et. al. Herpesvirus saimiri encoded a functional homolog of the human bcl-2 oncogene.//J. Virol.-1997.-Vol. 71.-P.4118-4122.

16 Thome M., Schneider P., Hofmann K., et al. Viral FLICE-inhibitory proteins (FLIPs) prevent apoptosis induced by death receptors.//Nature.-1997.-Vol. 386.-P. 517-521.

17 Shen Y., Zhu H., Shenk T. Human cytomegalovirus IE1 and IE2 proteins are mutagenic and mediate “hit-and-run” oncogenic transformation in cooperation with the adenovirus E1A proteins.//Proc. Natl. Acad. Sci. USA.-1997.-Vol. 94.-P. 3341-3345.

18 Zhu H, Shen Y, Shenk T. Human cytomegalovirus IE1 and IE2 proteins block apoptosis.//J. Virol.-1995.-Vol. 69. -P. 7960-7970.

19 Bresnahan W A, Albrecht T., Thompson E A. The cyclin E promoter is activated by human cytomegalovirus 86-kDa immediate early protein.//J. Biol. Chem.-1998.-Vol. 273.-P. 22075-22082.

20 Vermeulen S. J., Bruyneel E. A., Bracke M. E., et al. Transition from the noninvasive to the invasive phenotype and loss of alpha-catenin in human colon cancer cells.//Cancer Res.-1995.-Vol. 55.-P. 4722-4728.

21 Bumrns L. J., Pooley J. C., Walsh D. J., et al. Intercellular adhesion molecule-1 expression in endothelial cells is activated by cytomegalovirus immediate early proteins//Transplantation.-1999.-Vol. 67.-P. 137-144.

22 Cinatl J. Jr., Cinatl J., Vogel J. U., et al. Modulatory effects of human cytomegalovirus infection on malignant properties of cancer cells.//Intervirology.-1996.-Vol. 39.-P. 259-269.

23 Franco A, Jimenez L, Aranda I, Alvarez L, Gonzalez M, Rocamora N, Olivares J. Diffuse lymphoproliferative disease after renal transplantation and its relation with Epstein-Barr virus. Experience at one center//Nefrologia.-2002.-Vol. 22, N 5.-P. 463-469.

24 Barinskiy I F, Shabuladze A K, Kasparov A K, Grebenyuk V N. Herpes: Etiology, Diagnostics, Treatment. Moscow: Meditsina, 1986. 272 pp.

25 Barinskiy I F. Herpes Infections as Secondary Immunodeficit and Methods for Its Correction//The All-Union Institute of Scientific and Technological Information, Immunology series. Moscow, 1988. Vol. 22, pp. 126-146.

26 Kolomiets A A, Votyakov V I, Bikbulatov R. M. Generalized Herpes Infections: Facts and Concepts. Minsk. 1992. 350 pp.

27 Rancewicz Z, Kasprzycka M, Zagozdzon R, Wierzbicki P, Durlik M, Juskowa J, Gorski A, Paczek L. Effect of viral infection on T-cell apoptosis in allograft recipients//Transplant Proc.-2000.-Vol. 32, N 6.-P. 1403-1405

28 Bocharov A F, Kitsak V Ya, Bocharov E F, Trukhachev A A. The Herpes Simplex Virus. Novosibirsk. 1982. 322 pp.

29 Prigozhina V K, Chayka N A, Rakhmanova A G. Herpes Infection and AIDS: Recommendations for Doctors. Leningrad, 1990. 44 pp.

30 Rakhmanova A G, Prigozhina V K, Neverov V A. Infectious Diseases: A Handbook for General Practitioners. Moscow; St. Petersburg, 1995. 302 pp.

31 Sukhikh G T, Vanko L V, Kulakov V I. Immunity and Genital Herpes. Nizhniy Novgorod-Moscow, 1997. 221 pp.

32 Ketlinskiy S A, Kalinina N A. Immunology for the Doctor. St. Petersburg, 1998. 155 pp.

33 Berney T, Delis S, Kato T, Nishida S, Mittal NK, Madariaga J, Levi D, Nery JR, Cirocco R E, Gelman B, Ruiz P, Tzakis A G. Successful treatment of posttransplant lymphoproliferative disease with prolonged rituximab treatment in intestinal transplant recipients//Transplantation.-2002.-Vol. 74,N 7.-P.1000-1006

34 Patent application publication N US2003/0190602. Cell-based detection and differentiation of disease states/Norman J. Pressman, Kenneth S. Hirsch

35 Immunological methods of research./I Lefkovits and B Pernis, eds.//I., World.-1988.-pp. 456-458

36 D K Novikov, V I Novikova. Evaluation of immune status. Moscow-Vitebsk.-1996. -pp. 163-180.

1. A diagnostic method for the prediction of the development of oncological illnesses, in which samples of patients' tissues are taken, microdrugs are prepared, specific immunoglobulins are processed using an immunofluorescence method, and the percentage of fluorescing cells are determined and counted, distinguished by the fact that in the capacity of specific immunoglobulins, antiviral immunoglobulins are used and the quantity of cells infected by two or more viruses is determined.

2. A diagnostic method for the prediction of the development of oncological illnesses according to claim 1, in which the quantity of infected cells of any two or more viruses exceeds 50±10% in patients without signs of oncological pathology and a diagnostic conclusion has been reached on a high level of danger of oncological illness in connection with an ineffective immune system.

3. A diagnostic method for the prediction of the development of oncological illnesses according to claim 1, in which the quantity of infected cells of any two or more viruses exceeds 50±10% in patients with diagnosed oncological illnesses and a diagnostic conclusion has been reached on the low sensitivity of the cancerous tumor to chemotherapy and a perspective of quick tumor metastasis in connection with an ineffective immune system.

4. A diagnostic method for the prediction of the development and control of the effectiveness of the treatment of oncological illnesses according to claims 1-3, in which immunocytes from venous blood are used as patient tissue samples.

5. A diagnostic method for the prediction of the development and control of the effectiveness of the treatment of oncological illnesses according to claims 1-3, in which immunocytes from capillary blood are used as patient tissue samples.

6. A diagnostic method for the prediction of the development and control of the effectiveness of the treatment of oncological illnesses according to claims 1-3, in which erythrocytes from venous blood are used as patient tissue samples.

7. A diagnostic method for the prediction of the development and control of the effectiveness of the treatment of oncological illnesses according to claims 1-3, in which erythrocytes from capillary blood are used as patient tissue samples.

8. A diagnostic method for the prediction of the development and control of the effectiveness of the treatment of oncological illnesses according to claims 1-3, in which replicas of biopsy material from tumor tissues are used as patient tissue samples.

9. A diagnostic method for the prediction of the development and control of the effectiveness of the treatment of oncological illnesses according to claims 1-3, in which replicas of biopsy material from tissues surrounding the tumor are used as patient tissue samples.

10. A diagnostic method for the prediction of the development and control of the effectiveness of the treatment of oncological illnesses according to claims 1-3, in which replicas of biopsy material from healthy tissue are used as patient tissue samples.

11. A diagnostic method for the prediction of the development and control of the effectiveness of the treatment of oncological illnesses according to claims 1-3, in which cells from patient urocheras are used as patient tissue samples.

12. A diagnostic method for the prediction of the development and control of the effectiveness of the treatment of oncological illnesses according to claims 1-3, in which cells from patient salivary sediment are used as patient tissue samples.

13. A diagnostic method for the prediction of the development and control of the effectiveness of the treatment of oncological illnesses according to claims 1-3, in which various combinations of samples in claims 4-12 are used as patient tissue samples.

14. A diagnostic method for the prediction of the development and control of the effectiveness of the treatment of oncological illnesses according to claim 13, in which immunoglobulins against the type 1 human herpes virus are used as specific immunoglobulins.

15. A diagnostic method for the prediction of the development and control of the effectiveness of the treatment of oncological illnesses according to claim 13, in which immunoglobulins against the type 2 human herpes virus are used as specific immunoglobulins.

16. A diagnostic method for the prediction of the development and control of the effectiveness of the treatment of oncological illnesses according to claim 13, in which immunoglobulins against the herpes Zoster virus are used as specific immunoglobulins.

17. A diagnostic method for the prediction of the development and control of the effectiveness of the treatment of oncological illnesses according to claim 13, in which immunoglobulins against the human cytomegalovirus are used as specific immunoglobulins.

18. A diagnostic method for the prediction of the development and control of the effectiveness of the treatment of oncological illnesses according to claim 13, in which immunoglobulins against the Epstein-Barr Virus are used as specific immunoglobulins.

19. A diagnostic method for the prediction of the development and control of the effectiveness of the treatment of oncological illnesses according to claim 13, in which immunoglobulins against the type 6 human herpes virus are used as specific immunoglobulins.

20. A diagnostic method for the prediction of the development and control of the effectiveness of the treatment of oncological illnesses according to claim 13, in which various combinations of the immunoglobulins as per claims 14-19 are used as specific immunoglobulins.

21. A diagnostic method for the control of the effectiveness of the treatment of oncological illnesses, in which patient tissue samples are taken, microdrugs are prepared, specific antiviral immunoglobulins are processed using the immunofluorescence method, the number of fluorescing cells are determined and counted, distinguished by the use of antiviral immunoglobulins are used as specific immunoglobulins, the number of cells infected by two or more viruses before the beginning of treatment, in the process of treatment, and after treatment with the application of antiviral therapy are determined, and the dynamic of the change in the number of infected cells and their interrelationships are established:
when the number of infected cells decreases by more than 20±10%, the treatment is considered successful, whereas an absence of changes or an increase in the number of infected cells is considered an indication of unsuccessful treatment.

22. A diagnostic method for the control of the effectiveness of the treatment of oncological illnesses according to claim 21 that also applies antiviral drugs to cancer patients after their treatment with a combination of traditional methods and determines the change in the percentage of infected cells after treatment.

23. A diagnostic method for the control of the effectiveness of the treatment of oncological illnesses according to claim 21, in which a decrease in the number of infected cells of 20±10% or more is seen in a repeat diagnosis for two or more viruses and a conclusion is reached on the effectiveness of the therapy conducted.

24. A diagnostic method for the control of the effectiveness of the treatment of oncological illnesses according to claim 21, in which a decrease in the number of infected cells of 20±10% or more is seen in a repeat diagnosis for at least one of the viruses discovered by methods discovered earlier and a conclusion is reached on the ineffectiveness of the therapy conducted in that period and the necessity of changing the antiviral therapy scheme.

25. A diagnostic method for the prediction of the development and control of the effectiveness of the treatment of oncological illnesses according to claims 21-24, in which immunocytes from venous blood are used as patient tissue samples.

26. A diagnostic method for the prediction of the development and control of the effectiveness of the treatment of oncological illnesses according to claims 21-24, in which immunocytes from capillary blood are used as patient tissue samples.

27. A diagnostic method for the prediction of the development and control of the effectiveness of the treatment of oncological illnesses according to claims 21-24, in which erythrocytes from venous blood are used as patient tissue samples.

28. A diagnostic method for the prediction of the development and control of the effectiveness of the treatment of oncological illnesses according to claims 21-24, in which erythrocytes from capillary blood are used as patient tissue samples.

29. A diagnostic method for the prediction of the development and control of the effectiveness of the treatment of oncological illnesses according to claims 21-24, in which replicas of biopsy material from tumor tissues are used as patient tissue samples.

30. A diagnostic method for the prediction of the development and control of the effectiveness of the treatment of oncological illnesses according to claims 21-24, in which replicas of biopsy material from tissues surrounding the tumor are used as patient tissue samples.

31. A diagnostic method for the prediction of the development and control of the effectiveness of the treatment of oncological illnesses according to claims 21-24, in which replicas of biopsy material from healthy tissue are used as patient tissue samples.

32. A diagnostic method for the prediction of the development and control of the effectiveness of the treatment of oncological illnesses according to claims 21-24, in which cells from patient urocheras are used as patient tissue samples.

33. A diagnostic method for the prediction of the development and control of the effectiveness of the treatment of oncological illnesses according to claims 21-24, in which cells from patient salivary sediment are used as patient tissue samples.

34. A diagnostic method for the prediction of the development and control of the effectiveness of the treatment of oncological illnesses according to claims 21-24, in which various combinations of samples in claims 25-33 are used as patient tissue samples.

35. A diagnostic method for the prediction of the development and control of the effectiveness of the treatment of oncological illnesses according to claim 34, in which immunoglobulins against the type 1 human herpes virus are used as specific immunoglobulins.

36. A diagnostic method for the prediction of the development and control of the effectiveness of the treatment of oncological illnesses according to claim 34, in which immunoglobulins against the type 2 human herpes virus are used as specific immunoglobulins.

37. A diagnostic method for the prediction of the development and control of the effectiveness of the treatment of oncological illnesses according to claim 34, in which immunoglobulins against the herpes Zoster virus are used as specific immunoglobulins.

38. A diagnostic method for the prediction of the development and control of the effectiveness of the treatment of oncological illnesses according to claim 34, in which immunoglobulins against the human cytomegalovirus are used as specific immunoglobulins.

39. A diagnostic method for the prediction of the development and control of the effectiveness of the treatment of oncological illnesses according to claim 34, in which immunoglobulins against the Epstein-Barr virus are used as specific immunoglobulins.

40. A diagnostic method for the prediction of the development and control of the effectiveness of the treatment of oncological illnesses according to claim 34, in which immunoglobulins against the type 6 human herpes virus are used as specific immunoglobulins.

41. A diagnostic method for the prediction of the development and control of the effectiveness of the treatment of oncological illnesses according to claim 34, in which various combinations of the immunoglobulins as per pts. 35-40 are used as specific immunoglobulins.
